(19) United States
(12) Patent Application Publication
(10) Pub. No.: US 2026/0250616 A1
(43) Pub. Date: Aug. 27, 2026

(54) System and Method for Production of Advanced Therapy Manufactured Products (ATMPs)

(71) Applicant: Sedna Cell Care, Corp., Los Angeles, CA (US)

(72) Inventors: Rogelio Figueroa, Los Angeles, CA (US); Erin Stephens, Los Angeles, CA (US); Michael Benedict, Los Angeles, CA (US); Arturo Gonzalez, Los Angeles, CA (US); Manuel Santel, Los Angeles, CA (US)

(73) Assignee: Sedna Cell Care, Corp., Los Angeles, CA (US)

(21) Appl. No.: 19/394,771

(22) Filed: Nov. 19, 2025

Related U.S. Application Data
(60) Provisional application No. 63/722,477, filed on Nov. 19, 2024.

(51) Int. Cl. C12M 1/00; C12M 1/36; C12M 3/00; C12M 3/04; G01N 33/15

(52) U.S. Cl. CPC C12M 23/26; C12M 23/46; C12M 27/10; C12M 27/12; C12M 41/48; G01N 33/15

(57) ABSTRACT

A device and system for the medical processing of advanced therapy medicinal products. The system featuring a processing unit operating with a disposable fluid-container assembly constitutes a fully closed, self-contained manufacturing environment for cell-therapy production—including washing, enrichment, activation, genetic modification, expansion, media exchanges, in-process sampling, formulation, and final harvest of the produced cell therapy products.

### BACKGROUND OF THE INVENTION

### 1. Field of the Invention

This application claims priority to U.S. Provisional Patent Application Ser. No. 63/722,477 filed on Nov. 19, 2024.

This invention relates generally to a device and methods for the processing of advanced therapy manufactured products, also known as ATMP. More particularly, it relates to a computer controlled system having software operating to the task of automating ATMP and thereby improving outcomes.

### 2. Prior Art

The field of cell therapy has emerged as a revolutionary approach in the treatment of various diseases, particularly those that are otherwise difficult to address with conventional methods. Advanced Therapy Medicinal Products (ATMPs), which include gene therapies, cell therapies, and tissue-engineered products, have demonstrated substantial potential in regenerative medicine and personalized treatments.

However, the manufacture of these products remains a complex, time-consuming, highly labor-intensive, and costly process. These issues present a significant challenge to the widespread employment of ATMP therapies and the benefit to a wider array of patients from the wider availability of ATMP processing.

Current manufacturing processes for ATMPs involve intricate procedures that require highly specialized equipment and procedures, which are configured in operation to simulate the cellular environment of the human body. The existing devices and systems employable for ATMP products are often limited in their capacity to provide optimal, adjustable, and controlled conditions for cell culture and manipulation. As a result, the production of ATMPs frequently entails substantial operational expenses, which include high costs for materials, as well as costly trained labor and facility management.

Moreover, such ATMP processes can be extremely labor-intensive. As a consequence, they are highly prone to outcome variability. This is primarily due to the employment of differing workers with differing training and attention to detail. Such can often lead to inconsistent product quality, high rates of failure, and increased time to market.

With respect to the above, before explaining at least one preferred embodiment of the ATMP processing system herein, it is to be understood that the system herein is not limited in its application to the details of employment and to the arrangement of the components or the steps set forth in the following description or illustrated in the drawings. The various machine processing and software-enabled methods and steps of the herein disclosed ATMP processing system invention are capable of other embodiments, and of being practiced and carried out in various ways, all of which will be obvious to those skilled in the art once the information herein is reviewed.

Also, it is to be understood that the phraseology and terminology employed herein are for the purpose of description, and should not be regarded as limiting. As such, those skilled in the art will appreciate that the conception upon which this disclosure is based may readily be utilized as a basis for other devices and systems for ATMP processing. It is important, therefore, that the embodiments, objects, and claims herein be regarded as including such equivalent construction and methodology insofar as they do not depart from the spirit and scope of the present invention.

### SUMMARY OF THE INVENTION

The disclosed system herein provides for an ATMP processing of medicine based on genes, cells, or tissues. Such ATMP processes offer new opportunities for treating diseases and injuries. Further, such ATMP processing provides medical treatments which may be tailored to individual patients. By ATMP processing herein is meant gene therapy medicinal products, such as those that introduce recombinant genes to modify or replace faulty genes; somatic cell therapy medicinal products which utilize substantially manipulated cells or tissues for therapeutic purposes; tissue engineered products which contain or process engineered cells or tissues aimed at regenerating, repairing, or replacing human tissue, and other such processes as would occur to those skilled in the art.

In preferred embodiments, the processing unit together with the disposable fluid-container assembly constitutes a fully closed, self-contained manufacturing environment. All unit operations required for cell-therapy production—including washing, enrichment, activation, genetic modification, expansion, media exchanges, in-process sampling, formulation, and final harvest—are executed without opening the circuit or disconnecting any sterile interfaces. By eliminating open manipulations, the system maintains an ISO-class aseptic envelope throughout the entire process, thereby safeguarding product sterility, reducing contamination risk, and obviating the need for external biosafety hoods or Grade B clean-room interventions. The closed architecture further enables seamless automation of sequential steps under common software control, allowing the device to deliver a finished, release-ready Advanced Therapy Medicinal Product (ATMP) from a single setup.

Primary to the system herein is a processing unit which is configured for operative coupling with a fluid container, such as a fluid envelope. The processing unit is configured to replicate the optimal cellular environment found within the human body and to provide precise, versatile, and adjustable conditions for effective cell culture and manipulation over the duration of time of the ATMP process being facilitated by the processing unit.

While a fluid envelope is shown primarily as the fluid container herein for convenience, the fluid container may also be a container such as a commercially available culture bag or other vessel which is configured for operative coupling with the processing unit such including conduits for the ingress and egress of the required fluids and gasses, and which is configured such that it may be operatively coupled with a mount positioned on and operated by the processing unit.

The processing unit in operative coupling with a fluid container, such as a processing envelope, is configured to constantly monitor the contents of the processing container, especially at a known time, elapsed time points, or specific time points during the aggregate duration of the process being performed by the processing unit.

The processing unit preferably operates substantially autonomously once a fluid container has been appropriately populated with fluid and the other contents required for the process intended. That is to say AI or other software running in electronic memory, operatively communicating with a computing device, operates to perform the automation of repetitive tasks, visual and sensor data process monitoring of the container contents, and based on data captured in quality control checks at time points during an ongoing ATMP process, comparing a current discerned status of the contents of the fluid container to known data sets of the same process at substantially the same time point to ascertain a current matching or in compliance with the known process.

Based on a comparison of captured environmental parameters and digital images of a particular ATMP regimen or process being monitored by the processing unit operatively coupled with the fluid container, at one or more time points during the duration of the ATMP process being monitored and facilitated by the processing unit, to known matching or appropriate environmental parameters and digital images of the ATMP process at the captured time point, the system herein using the processing unit may continue the process if the review shows a match of discerned images and/or environmental parameters at the time point, or it may intervene and take remedial action and/or inform a technician where the discerned elements at that particular time point do not match what are known to be required.

The processing unit is configured to provide multiple means for physical processing of the contents of an operatively coupled fluid container. Such include subsystems for providing rotation of the fluid container, actuation of the container, material transfer into and from such a fluid container, and ongoing high-resolution imaging of the contents of a fluid container. The processing unit is configured for operative coupling with the fluid container of choice. By operative coupling is meant that the processing unit has a mount for coupling the fluid container, and so mounted thereafter provides the mechanical, pneumatic, and imaging functions needed for the duration of a range of cell-based processes essential for advanced cell therapy manufacture. The rotation of the fluid container and the mechanical actuation thereof, along with the components for material transfer, ongoing image capture and comparison, and parameter capture and comparison, enable the processing unit to have precise control over critical unit operations during the duration of the ATMP or cell handling regimen being performed.

The rotation system allows the processing unit to rotate an operatively coupled fluid container upward up to substantially 270 degrees, in both clockwise and counterclockwise directions around a central axis. The rotation system includes a housing assembly and a column assembly.

The housing assembly has a sturdy frame, or housing, which supports a back light adjacent to a solid transparent backing. The provision of such a back light adjacent to the transparent backing provides for ongoing, consistent illumination of the fluid container and contents and visibility thereof to operatively positioned video capture components. The housing assembly also provides structural support for the actuation subsystem, which includes actuators that may move over the flexible culture envelope efficiently and without sway.

The column assembly has a motor coupled thereto at a base, and has a rotation gear and a pivot assembly at the top on each side, which may be rotationally coupled to the housing. The rotation gearing connected to the controllable motor enables controlled rotation of the housing. An angular encoder or accelerometer mounted on the housing or calibrated to the rotation gearing provided measurements of substantially the exact angle of rotation at any given moment in time. This allows the housing and engaged fluid container to be repositioned as needed without placing undue tension on connecting fluid and gas lines, which may be operatively coupled with the fluid container, such as the noted flexible envelope.

The actuator subsystem may be controlled by software operating to manage the ongoing mechanical manipulation of the cell culture fluid envelope. This actuator system employs one or both of rollers and a paddle to thereby communicate low-shear cell manipulation to the fluid container, such as the envelope. This manipulation creates compartments within the envelope when pressed against the transparent backing to which it is adjacently mounted.

Magnets, particularly paramagnetic beads which may be magnetically activated by an electromagnet within the paddle, may also be employed for any required cell separation processes. The subsystem may include load sensors on the rollers to provide a measurement of the force thereof against the envelope. It may also include encoders or location sensors to monitor roller position during any surging, heaving, and rolling actions imitated by the software operating the system.

The roller component may include two movable rollers (upper and lower), which are moveably positionable to perform heaving, surging, and rolling actions. Heaving actions shift the roller up and down (in a vertical orientation) or side to side (in a horizontal orientation), aiding in compartment volume adjustments or moving material out of specific sections. Surging actions move a roller closer to or away from the fluid-containing envelope, creating compartments or narrow flow cross-sections in the interior chamber that can precisely control fluid and cellular movement. The extent of the surge is monitored via an encoder, while the roller load sensor measures force. Such initiated rolling actions operate to shift the roller to shift the position thereof while in a full or partially closed state, thereby guiding fluid flow or mixing within the interior chamber of the fluid container envelope.

Where the paddle is included, a single paddle, attached to a paddle rail, may be operated in two degrees of freedom—surge and heave. The paddle operation can thus form narrow channels near the fluid envelope, thereby operating for controlling fluid dynamics and cell distribution. The paddle may include a flat face for forming these narrowed pathways. The paddle may also be equipped with an electromagnet which may be activated by software operating to provide such activation for magnetic bead separation.

Still further, the paddle may be configured to house a microscope for real-time imaging of specific depths of field of the fluid contents of an adjacent fluid container. It may also have optical sensors thereon operating to connect and communicate with in-envelope measurement systems.

The processing unit preferably includes a material transfer assembly for communication of required fluids and materials into and out of the interior chamber of an engaged fluid container, such as the fluid envelope. This material transfer assembly may be a component of the actuator subsystem or may be an adjacent component as long as it is operatively coupleable to the fluid container to provide the required material transfer during the chosen ATMP process.

The material transfer assembly comprises the material transfer base and has valve actuator pins or controls therefor. The material transfer base is a sturdy frame, such as metal, and is configured to provide the required structural support for the actuators, which are operable for regulating both gas and liquid valves on the fluid container envelope. In operation, which may be controlled by software operating to the task, as with other operable components herein, a valve actuator pin may be translated inward and outward to thereby open or close these valves. The actuator may also travel vertically to position itself overhead of the valves or secure lines. Through these valve actuator pins, the system running software operating to the task, can control inflow and outflow of fluids and gases within the interior chamber of the fluid container, such as a flexible envelope.

Additionally preferred in the system herein is an imaging subsystem having a plurality of imaging components. Such imaging components may include a macro visualization camera, a micro visualization camera (microscope), and an illumination assembly.

The macro visualization camera is operatively coupled into a position to capture digital images of an overall view of the fluid container envelope, including the rollers and paddle noted above. The macro visualization camera thus provides wide-field observation and digital imaging of the fluid container contents, which may be employed for detecting foreign particles therein, observing liquid volume changes therein, and monitoring cell settling or aggregation within the container. The captured digital images at time points during the duration of the cell processing regimen, may be compared using software adapted to the task, with a database of digital control images at a time point, to determine if the current captured digital images show the process proceeding in an optimum fashion, or, to determine a variance at the time point from an optimum processing. Where a variance is determined, software operating to discern process adjustments employable may initiate such to bring the process back to an optimum processing for the time point, which may be confirmed by recapturing digital images subsequent to the adjustments to determine the process has been returned to what is expected at the time point of visual inspection.

The micro visualization camera may be coupled to such as being embedded in the paddle. The micro visualization camera provides close-up digital imaging of cellular morphology, clumping, and other microscopic changes, which may be visual at the chosen time point in the process. The micro visualization camera moves along with the paddle vertically along the length of the fluid container envelope. The captured close-up imagery also supports dynamic cell counting, thereby allowing software operating to the task, or operators, to estimate a current cell density without removing samples from the fluid container as would be required with conventional systems.

The noted illumination assembly has a transparent backing and an adjacent back light. Used in combination with digital imaging cameras, clear, high-contrast visualization of the fluid container envelope and the contents within the interior chamber is obtainable throughout the duration of the processing regimen. This allows for the capture of digital images, which may be compared with libraries of digital images of such container contents for a particular processing regimen, to determine if the imagery at a time point is substantially a match to stored imagery for the process.

The fluid container, such as a flexible envelope shown herein, may be easily engaged with the processing unit by opening a door to the housing, thereby exposing the solid transparent backing within the housing assembly. Connectors between the envelope and the transparent housing, such as positioning pins at each corner, may be employed to removably secure the envelope in place. Closing operates to align the valve actuators with the valves upon the fluid container envelope. Such allows the valve actuators to be controlled to automate the opening and closing of the coupled valves, which communicate with the interior chamber.

Thereafter, an operator or software operating to the task may operate to position the rollers and paddle to form sections in the fluid container, back defining compartments for separate reactions or manipulations. These compartments may be isolated to hold only liquid, only air, or combinations of liquid and air. By selectively positioning the paddle and rollers by an operator or software operating to that task, distinct processes can occur simultaneously without compromising the overall cell culture or process environment.

The operations of the processing unit and system herein as noted, may be controlled by a human operator or by software operating to perform each of the noted steps or tasks herein, or by combinations of human and software-controlled operations.

In order to initiate cell processing in the interior chamber of the fluid container envelope, it may first be pre-inflated with gas by closing one of the liquid valves and opening the other to thereby allow controlled gas inflow. This ensures a more uniform distribution once liquid is introduced, enhancing mixing and dispersion. Alternatively, lowering the external pressure around the fluid container envelope can achieve a similar effect.

The interior chamber of the fluid container envelope may be filled with fluid and other medium from the bottom thereof by valve actuators opening one liquid valve for liquid inflow and partially closing the other to allow air escape from the interior chamber of the fluid container envelope at a controlled rate. Such a process helps maintain a steady flow, minimize splattering, and create a stable, homogeneous process environment.

The material transfer system operates the valve actuators to open, close, or partially open the liquid and gas valves to control and coordinate the movement of liquid, air, and gases within the interior chamber of the fluid container envelope. As with all other actions herein it may be operated by software operating to that task, or by a human operator, or a combination of both.

The fluid container envelope may operate to dispense liquid from the interior chamber through the bottom thereof by activating the valve actuator to open a liquid valve for outflow therethrough while opening the other valve for gas backflow. This prevents interruptions in fluid outflow by balancing internal pressure.

When dispersions need time to settle, the processing component may be fixed in place at a specific angle with concurrent constant conditions, thereby allowing time-dependent settling and diffusion. This operative method is useful for concentrate collection or decanting after cells have settled.

The actuation subsystem may operate to generate pulsing through actuation of the roller for small, gentle, repetitive back-and-forth motion of the roller. This roller movement offers mild mixing and resuspension of cells or particles without disturbing the bulk of the material.

Caressing action by the system employs actuation of the roller for a stroking motion of the roller along the fluid container envelope corresponding to the envelope height. It is used to achieve bulk mixing with minimal shear forces, helping to gently resuspend cells within the interior chamber of the fluid container envelope and to thereby break apart fragile clumps.

### Squeezing

Squeezing action by the system herein is performed by a stroking of the first roller in the direction of a second roller positioned such that the interior chamber of the fluid container envelope is substantially sectioned off. This forms a narrow gap or pathway for liquid to pass through. By pushing liquid through this constrained pathway, material clusters or clumps within the liquid are broken apart effectively, thereby promoting homogeneity.

Liquids containing particles can be introduced into the interior chamber at the bottom of the fluid container envelope by energizing the appropriate valve actuator to open one liquid valve for inflow and partially closing the other liquid valve to control air outflow. This method helps maintain steady flow of liquid and minimize splattering, leading to uniform dispersion.

The Actuation Subsystem can operate to create a wiping motion of the roller in the contact with the transparent backing to reduce the volume of a sectioned area. As the roller moves, an opening of a liquid valve releases pressure from the diminishing volume.

The stem may also provide volume closing. By moving a roller into contact with the transparent backing, the system can section off a segment of the interior chamber of the fluid container envelope. Venting of gas from that space allows the volume of that segment to decrease, effectively isolating a smaller portion of the fluid container envelope.

The system may operate to impart magnetic control by turning the electromagnet in the paddle on and off for a process in combination with paramagnetic beads. In conjunction with antibody-conjugated beads, targeted cell types expressing specific antibodies can be isolated.

The system may also operate to provide cell selection. Initiating a gentle intermittent pushing by the rollers, or a rocking of the fluid container envelope, operates to flow liquid over the magnet. This allows specific cells bound to paramagnetic beads to be captured.

Where the system is employed with a two-chamber fluid container having a liquid reservoir with a liquid space and a gas reservoir and having gas space and having a gas-permeable membrane therebetween, the system can employ gas filling. The gas space or gas reservoir side of the fluid container envelope can be filled with a predetermined mixture of gases. Because the membrane between the gas space and the liquid space is gas-permeable, this allows for precise control over air, oxygen, carbon dioxide, and other gases that diffuse into the liquid.

### Slow Rotation

The rotation subsystem can be operated to orient the fluid container envelope or bag at various inclinations. This acts to modify the liquid/gas interface area without significantly resuspending cells. This rotation action and orientate helps manage settling or layering of dispersed components.

In another software or user-controlled operation of the system herein, the two-chambered fluid container envelope or bag may be rotated to form an increased diffusion area. Positioning the fluid container envelope in a substantially horizontal orientation, with the liquid space facing upward, maximizes the contact area between the gas-permeable membrane and the liquid, thereby enhancing gas diffusion rates.

Still further, the rotation subsystem can be operated to create a rocking motion, by rotating the fluid container envelope back and forth at a preset angle and at a preset speed—typically 6-10 degrees at 10-25 rocks per minute. This gentle agitation helps ensure oxygenation and nutrient distribution while maintaining cell integrity.

As noted, the imaging cameras of the system are employable for inspection of the contents of the fluid container envelope at time points during the duration of the processing regimen. Additionally, several verification processes may be performed using the noted imaging and sensing capabilities. First imaging by the digital camera is employable to measure the volume of liquid in the fluid container envelope. The images are also employable to evaluate mixing and to identify large clumps or clusters within the liquid. Still further, the microscope digital images are employable to identify clumps or particle agglomeration, enabling microscopic dynamic studies. The captured microscope images at any time point may be compared with a library of digital images in memory to ascertain clumps or particle agglomeration.

In an additional capacity, the system is employable for determination of pH/dissolved oxygen determination. Connectors on the paddle may be employed to interact with sensors located on the interior of the fluid container envelope to obtain real-time readings of pH and dissolved oxygen.

In operation, the user may choose the desired ATMP regimen or process for the system to perform. Each such ATMP regimen will have a known movement and manipulation of the contents of the interior chamber of the fluid envelope and multiple vertical and horizontal positioning thereof during the chosen process processing. Digital imaging components noted capture digital images at time points in the chosen process, provide for ongoing imaging of the contents of the processing fluid envelope to allow the system which are comparable to stored digital images for matching to discern a properly proceeding ATMP process. Should a digital image comparison show the envelope contents out of optimum from that which is preferred, software operating to take remedial action using changes to movements or environmental parameters known the cause the appropriate remedial action, and/or inform a technician, adjust the operation of the processing unit as needed to maximize the ATMP product outcome.

The processing envelope is configured to standardize the components employed for the processing of human or other cells for the ATMP regimen of choice. The processing fluid envelope, when operatively coupled with the processing unit, is adapted to simulate the optimal cellular environment found within the human body, during processing of the cells within the processing envelope.

The processing unit into which the processing envelopes are operatively coupled is configured to provide the processing envelope multiple positioning. That is to say, the processing unit is mechanically configured to provide container movements and move the envelope between a first vertical position, an upside-down second vertical position opposite the first vertical position, and a horizontal position during the processing of the cells within.

Further, the processing unit is equipped with imaging components configured to continuously image the contents of the interior chamber of the processing envelope during time points or stages of the processing. Such imaging components may include a camera for capturing full-sized imaging of the contents, as well as a digital microscope configured to capture microscopic images of the envelope contents during processing. Still further, a thermal imaging camera, such as an FLIR may be employed to provide thermal imagery of the processing envelope contents of media and cells and the like during processing.

The system for ATMP processing herein integrates advanced features that operate to reduce the time and costs associated with ATMP manufacturing. To that end, the system includes a patient-centric and cell-conscious design that helps ensure that each therapy is produced under optimal conditions, thereby improving product consistency and efficiency.

By streamlining the manufacturing process and minimizing resource expenditure, the cell processing system has the potential to make advanced therapies more accessible and cost-effective, thereby supporting the broader adoption of these promising treatments in clinical practice. The disclosed processing envelope-based system provides a crucial step toward meeting the ever-evolving needs of the cell therapy industry.

The cell processing system herein is configured for widespread use, such as in hospitals, research sites, treatment development sites, and even patient homes. To that end, the cell processing system herein will reduce the cost of treatment as well as increase the availability of cell and gene therapy treatments to more patients around the world.

Using the processing unit and fluid container envelope system herein, the manufacture of employable advanced therapies is provided by the ability of the system to emulate the optimal cellular environment found within the human body. In this manner, the system herein ensures that the cells being processed remain in an optimal environment throughout the manufacturing process.

The processing envelope may be configured as a single-chamber fluid container envelope or a multi-chamber envelope, which provides a fully enclosed, sterile processing container capable of executing multiple unit operations for a variety of advanced therapies. The processing unit to which the processing envelope automatically provides mechanical and pneumatic actions to the contents of the liquid and gas spaces of the interior chambers of the processing envelope to facilitate the execution of unit operations for advanced therapies. By operative coupling, herein is meant that the processing envelope is removably engaged with the processing unit, and any and all tubes or conduits required for processing of cells within media located within the processing envelope are connected.

During such processing, the system herein is configured to provide delicate handling of cells through a low-shear stress mixing within the interior chamber. This low-shear stress mixing operates to mimic the conditions found within the human body. This gentle approach minimizes cellular damage during the process period and also maintains the viability and potency of the cells. The system's ability to maintain controlled environmental parameters, which include at least precise air pressure, fluid envelope temperature, and gas composition within the fluid envelope and adjacent to it, further ensures that cells are cultured and manipulated under optimal conditions, reducing the risk of damage.

The ATMP processing system herein is highly adaptable due to its ability to adjust each process based on patient-specific conditions. Using the processing fluid container envelopes, which are operatively coupled to the processing unit, the system can adapt to patient-to-patient variability, ensuring maximum yield and potency of the cell therapies. The ATMP processing system has the capability to simulate various cellular environments, fine-tune gas compositions, and manage the cell culture conditions in real-time, allowing it to respond effectively to the unique individual requirements of each patient's cells.

The standardized and automated configuration of the ATMP processing system operates to significantly reduce the time frame for producing cell therapies from those of conventional systems. The employment of processing envelopes that operatively couple with a small processing unit minimizes the need for human intervention, optimizing material transfers, and avoids cryopreservation, thereby accelerating the manufacturing process. Additionally, continuous improvement based on real-time monitoring and risk management reduces the likelihood of failed batches, ensuring that therapies are produced efficiently and on schedule.

The cell processing system herein is compact, allowing for use in many locations where cell processing heretofore could not be accomplished. The simplified operation and avoidance of cryopreservation of the system herein makes it possible to produce approved therapies closer to the point of care. This increases the availability of these therapies, as they can be manufactured more rapidly and delivered to patients without the delays associated with centralized manufacturing and extensive transport.

The cell processing system herein provides advanced and ongoing real-time monitoring and control of the processing for the duration of such for the contents of the processing fluid envelope. This is accomplished through the inclusion of the noted integrated imaging subsystem, which provides macro and micro imaging of the contents within the interior chamber of the processing fluid envelope during the entire duration of the chosen process. The system herein also includes integrated dissolved oxygen and pH meters in the processing envelope, thereby enabling real-time assessment of cell health, pH levels, oxygenation, clumping, and growth.

Employing this real-time assessment at time points along the duration of a chosen ATMP process, from a menu of such processes, the system provides an ability to initiate adjustments during the processing based on these measurements by human input or through software operating on a computing device, which operates to each task or adjustment. Such adjustments, for example, and in no way limiting, can include media addition or changes in mixing conditions within the envelope to thereby ensure precise control over the cell culture environment therein. Further, the real-time digital imaging and other assessments provide users and software operating to the tasks, a real-time feedback loop for process monitoring, thereby enhancing the quality and consistency of the final cell therapy product.

In all modes of the system and steps herein noted where automated or software enabled controls and operation are employed, the system may use onboard computers or may employ network accessible servers or computers having accessible electronic memory for storage and retrieval of electronic software operating software and/or database information operating to perform the stated tasks or steps required of the processing unit during processing of the contents of processing envelopes.

Any adjustments required to the contents of the processing envelope, such as media and cells therein, may be determined and thereafter performed by the software. Software, running in electronic memory, may operate to the tasks of performing each step, or adjustment, or task, or calculation herein.

As to electronic memory or computer-readable media for the system herein, any combination of one or more computer-usable or computer-readable media, be it transitory or non-transitory, may be employed for operation of the software and assessment system herein. Such, for example and in no way limiting, can include computer-readable media and may include one or more of a portable computer diskette, a hard disk, a random access memory device, a read-only memory device, an erasable programmable read-only memory (EPROM or Flash memory) device, a portable compact disc read-only memory device, an optical storage device, and other electronic memory magnetic storage devices. Software or computer program code for carrying out the individual and sequential operations of the cell processing system may be written in any combination of one or more programming languages.

The steps or method of operation and/or execution of the various modes and tasks of the cell processing system herein may be illustrated as blocks or steps in the drawings, which may represent one or more sequences in the operation of the steps and assessments in the system herein. These operations or steps can be implemented in hardware, software operating to process input data to accomplish the task or step, or a combination thereof.

With regard to software operating to a task or steps or assessments indicated in the system herein, such represents computer-executable instructions stored upon one or more transitory or non-transitory computer-readable storage media, which, when executed by one or a plurality of processors, will operate to perform the recited task, assessment, operation, or step. Computer-executable instructions, in general, include routines, programs, algorithms, data structures, and the like, which are configured to perform particular functions or to implement particular abstract data types or steps noted.

It should be noted that the sequence in which the steps of the system herein are described or depicted for operation of the processes of the system is not intended to be construed as a limitation. It should be understood that any number of the described or designated steps can be combined in any order and/or in parallel to implement the described and depicted assessments and processes. In some modes of the system herein, one or more steps can be rearranged or omitted entirely. Still further, the software-enabled steps in the system herein can be combined in whole or in part with each other or with other steps or methods.

With respect to the above description, before explaining at least one preferred embodiment of the system and method for ATMP processing, it is to be understood that the invention is not limited in its application to the details of operation, nor the arrangement of the ATMP processing components, or the steps set forth in the following description or illustrations in the drawings. The various methods of implementation and operation of the system and method herein are capable of other embodiments and of being practiced and carried out in various ways, which will be obvious to those skilled in the art once they review this disclosure. Also, it is to be understood that the phraseology and terminology employed herein are for the purpose of description and should not be regarded as limiting.

Therefore, those skilled in the art will appreciate that the conception upon which this disclosure is based may readily be utilized as a basis for the designing of other methods and systems for ATMP processing. Therefore, the objects and claims herein should be regarded as including such equivalent construction, steps, and methodology insofar as they do not depart from the spirit and scope of the present invention.

It is an object of this invention to provide a standardized system for each of a plurality of different ATMP processes that minimizes the requirement of human operation and intervention.

It is a further object of this invention to provide such an ATMP system, which employs standardized fluid containers, such as processing fluid envelopes, which are operatively coupled with the processing unit.

It is a further object of the invention to provide such a system wherein image capturing components for capturing digital imagery are included to monitor the process at chosen time points for a duration of a chosen process and allow for human or computer-initiated input as to steps or changes in processing as required.

These, together with other objects and advantages, which will become subsequently apparent, reside in the details of the components and operation of the ATMP processing system herein as more fully hereinafter described and claimed, reference being had to the accompanying drawings forming a part thereof, wherein like numerals refer to like parts throughout.

Further objectives of this invention will be ascertained by those skilled in the art, as brought out in the following part of the specification, wherein a detailed description is for the purpose of fully disclosing the invention without placing limitations thereon.

### BRIEF DESCRIPTION OF DRAWING FIGURES

The accompanying drawings, which are incorporated herein and form a part of the specification, illustrate some but not the only or exclusive examples of embodiments of system components and/or steps of the ATMP processing system herein. It is intended that the embodiments and figures disclosed herein are to be considered illustrative of preferred modes of the system rather than limiting.

Further, it should be noted that the drawings are not necessarily to scale, and some features may be exaggerated or omitted for clarity.

In the drawings:

FIG. 1 depicts a perspective view of a processing unit herein showing a housing rotationally coupled to posts and having a pivoting section engaged to a first section of the housing for providing access to an interior cavity for operative coupling of a fluid container, such as an envelope thereto.

FIG. 1A steps in the software-controlled operation of the processing unit and system herein when employed for various cell processing regimens.

FIG. 2 depicts examples of some processing envelopes employable with the processing unit and autonomous system herein.

FIG. 3 shows side views of a dual-chamber envelope fluid container employable with the processing unit.

FIG. 4 shows side views of single-chamber envelope fluid containers employable with the processing unit.

FIG. 5 depicts the operative coupling of a bag or processing envelope upon a transparent backing which is coupled to an operative position in the processing unit.

FIG. 6 shows the positioning of valve actuators controlled by the processing unit in operative coupling with valves of a fluid envelope.

FIGS. 7-8 show components of the processing unit.

FIG. 9 shows cameras of the processing unit and a backlit surface for illumination of the contents of a coupled fluid envelope.

FIG. 10 depicts the rotational components where the housing may be rotated to differing angles during a processing regimen.

FIG. 11 depicts the rotation of the housing by the rotation system to various angles relative to the support surface.

FIG. 12 shows the angled positioning of the operatively coupled fluid envelope as required during any processing regimen.

FIG. 13 shows the positioning of a microscopic camera upon a translating paddle.

FIG. 14 shows a depiction of the capture of microscopic digital images of the contents of a coupled fluid envelope during a chosen processing regimen.

FIG. 15 depicts side views of empty fluid envelopes prior to use in the system.

FIG. 16 shows a filling of an interior chamber of the fluid envelope with air or gas.

FIG. 17 depicts an interior chamber of a fluid envelope enlarged by gas communicated therein at pressure, and air therein being evacuated.

FIG. 18 depicts the fluid envelope filled with gas at pressure with the valve closed.

FIG. 19 shows another view of the inflation of an interior chamber of a fluid envelope where gas is communicated therein through a lower valve.

FIG. 20 shows a filling of a portion of the interior chamber of a fluid envelope through liquid input through a valve in the lower end while air or gas is evacuated from the upper end.

FIG. 21 depicts the fluid and cell mixture held in the interior chamber with the valve closed upon completion of filling.

FIG. 22 shows the dispensing of the liquid within the interior chamber of the fluid envelope by opening a lower valve for fluid flow and an upper valve for input of gas or air.

FIG. 23 shows the settling of solids within the fluid held in an interior chamber during wait times with non-movement or manipulation of the fluid envelope.

FIG. 24 depicts imparting of a translating pulsing by horizontal movement by a roller to the fluid within the interior chamber.

FIG. 25 shows the communication of a caressing contact of the roller by a combined horizontal and vertical movement of the roller.

FIG. 26 shows the squeezing movement of the roller to segment the interior chamber and fluid therein into sections.

FIG. 27 depicts the communication of additional fluid into the interior chamber through a valve at the lower end of the fluid envelope.

FIG. 28 shows an action to reduce the volume of gas in the interior chamber by a sealing translating contact of the roller sandwiching the fluid envelope between the roller and transparent planar surface.

FIG. 29 shows the venting of liquid from an overhead valve by movement of the roller from a lower position to an upper position.

FIG. 30 depicts the action of increasing the fluid within the interior chamber through the formation of a vacuum therein.

FIG. 31 shows a side view of both a single chamber or a dual chamber fluid envelope employable with the processing unit, having fluid filling a portion of the interior chambers.

FIG. 32 those the employment of the paddle and a pair of rollers to evacuate the gas and form fluid filled segments within the fluid chambers.

FIG. 33 shows the contact of the paddle and rollers on opposing sides thereof with the fluid container to form segments within the fluid chamber having fluid and gas therein.

FIG. 33 shows the contact of the paddle and the rollers of the system to form a pair of gas-filled segments and a pair of fluid-filled segments within the fluid chambers.

FIG. 35 shows an action of the system herein to section off the fluid within the interior chamber from the gas therein.

FIG. 36 the communication of gas to an upper portion of the interior chamber while the fluid is isolated by the roller in a lower portion thereof.

FIG. 37 shows the processing unit imparting a low rotation to the fluid envelope during a processing regimen.

FIG. 38 depicts he horizontal positioning by the rotation system of the fluid envelope.

FIG. 39 shows opposing angles of inclination imparted by the rotation of the housing of the processing unit.

FIG. 40 depicts the employment of the digital camera to capture digital images of the fluid contents of the interior chamber for the determination of volume.

FIG. 41 shows the camera of FIG. 4 capturing imagery employable to determine the homogeneity of the contents of the fluid in the interior chamber.

FIG. 42 shows an example of the paddle-mounted microscopic camera imaging the fluid contents to determine clumping.

FIG. 43 shows the employment of the paddle with an optical sensor thereon for the determination of PH and dissolved oxygen.

FIGS. 44-45 show the employment of an electromagnet to capture selected cells.

### DETAILED DESCRIPTION OF THE PREFERRED EMBODIMENTS OF THE INVENTION

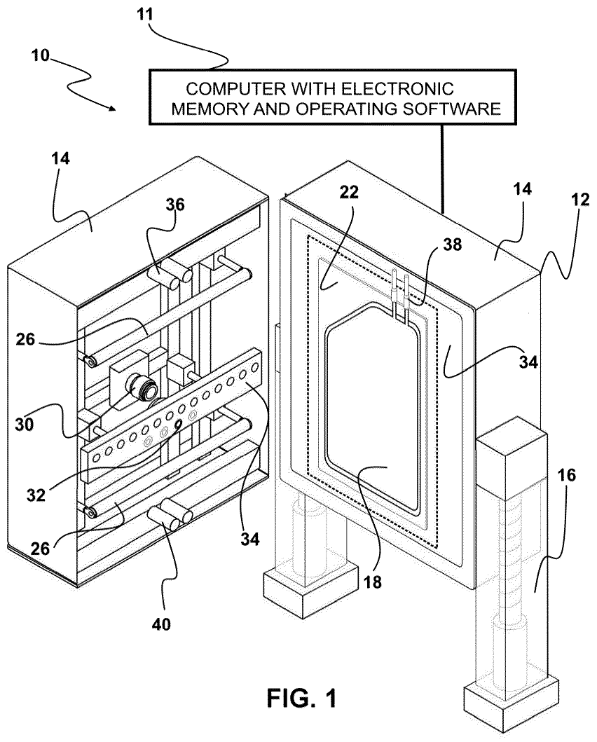

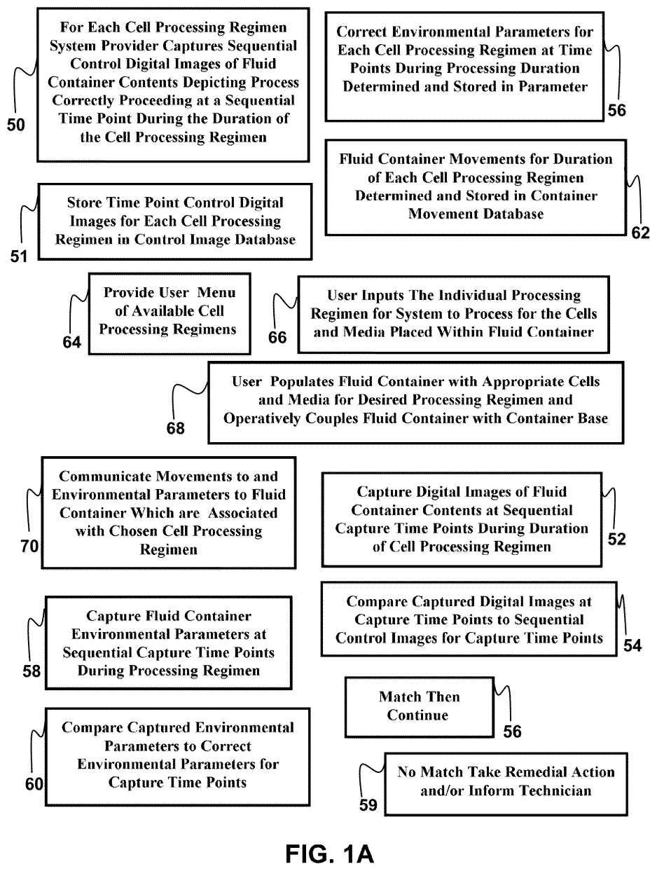

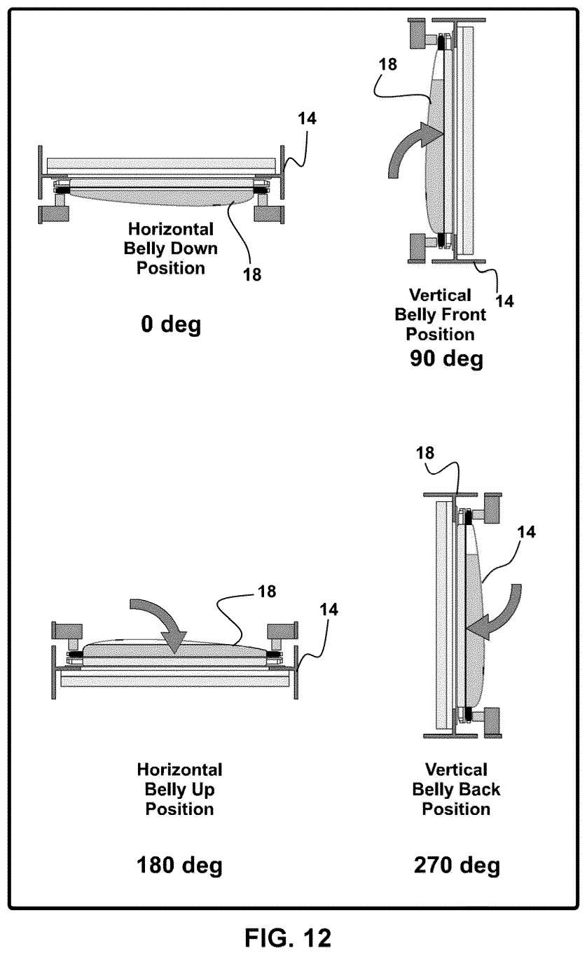

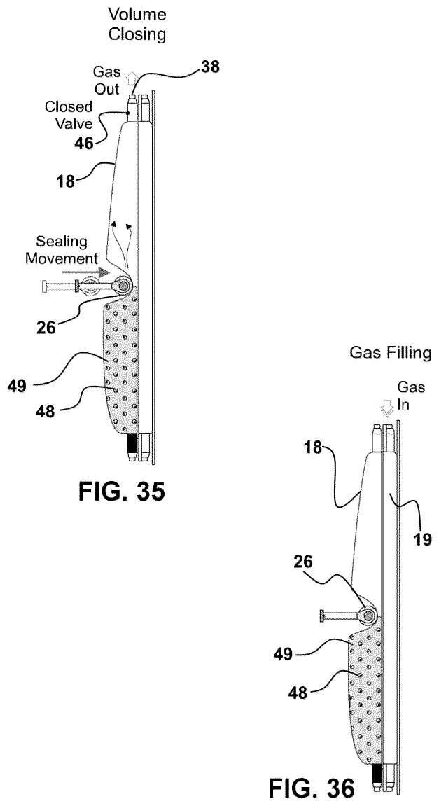

Referring now to the method and cell processing system 10 for ATMP products, herein shown in simple format by the depictions of FIGS. 1-45. It should be noted that where operations or movements of different components of the processing unit 12 to enable the operation of the processing system 10 herein are initiated, to manipulate the fluid contents of an interior chamber of a single chamber or dual chamber fluid envelope herein, each such movement or operation may be initiated and controlled by software operating to perform the noted movement or operation.

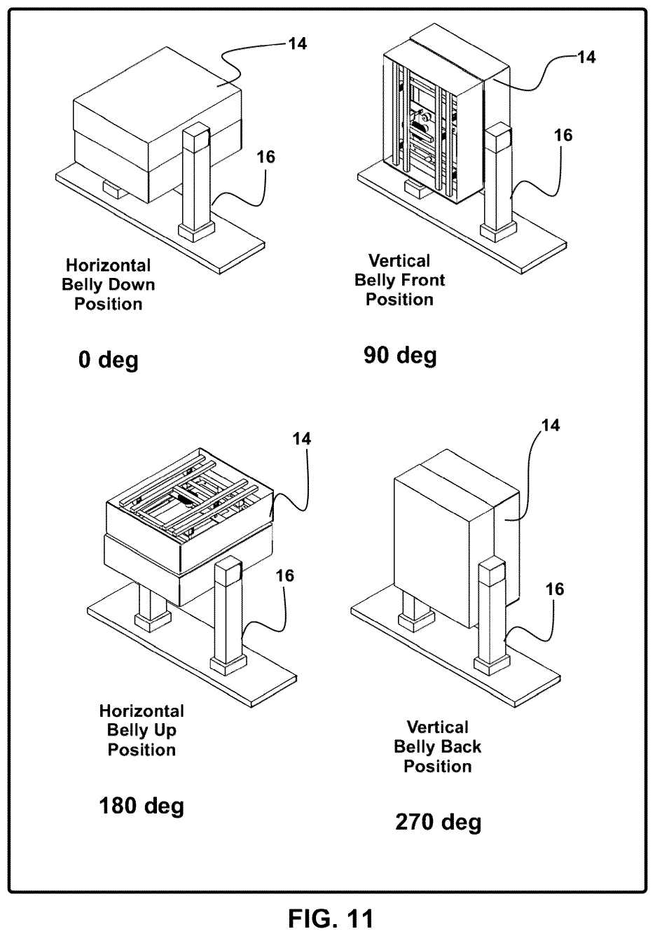

In FIG. 1 there is shown the processing unit 12 having a housing 14 having a first section thereof and a second section in a pivoting engagement, such as with a hinge to the first section, for allowing access to an interior cavity 15. A latch or other connector may be employed to keep the second section closed position, such as in FIG. 11.

The first section of the housing 14 may be rotationally coupled to one or a plurality of supports 16 such that the housing 14 and interior components and contents of an interior cavity 15 may also be rotated to differing angles relative to the support surface on which the housing 14 and support or supports 16 are positioned. Software running in electronic memory of a computer 11 may be used to operate the device and to perform the multiple operations herein.

As shown, a fluid envelope 18 of choice may be operatively coupled with the processing unit 12 such as by engagement to and against a substantially transparent planar backing 22 which is removably coupled to a position adjacent an illumination assembly 24 which may have a light emitter and illumination sheet or an LED sized to communicate light through the substantially transparent planar backing 22 and the contents of any interior cavity 23 of the substantially transparent fluid envelope 18. By substantially transparent planar backing 22 is meant herein, that light will pass through the material forming the transparent backing to be employed for illumination of the contents of the interior cavity 23 of the envelope.

One or a pair of rollers 26 are shown, which are mounted to powered tracks 28 for imparting vertical movement to the rollers 26 together or independently. By powered tracks herein is meant that a motor 29 is engaged with the track 28 such that controlled translation of the attached rollers 26 vertically can be imparted by energizing the motor 29 to move the rollers 26 up or down. This can be done in a number of ways, such as by reversing the voltage supplied to the motor 29 to change directions. Additionally shown are a digital camera 30 and a microscopic digital camera 32, which is in a recessed positioning with a translating paddle 34 which is also moveable by the powered track 28 in a vertical translation.

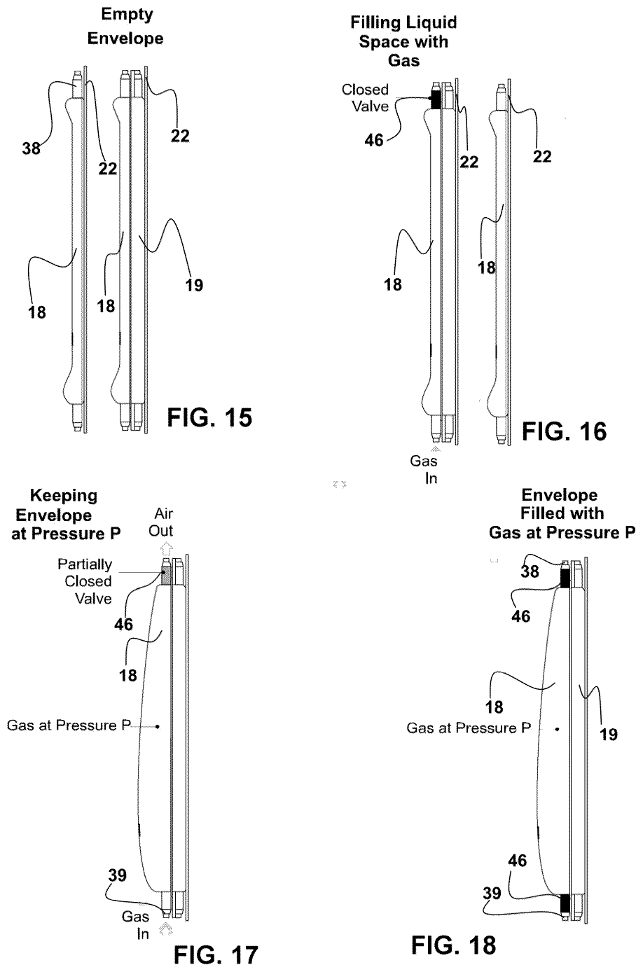

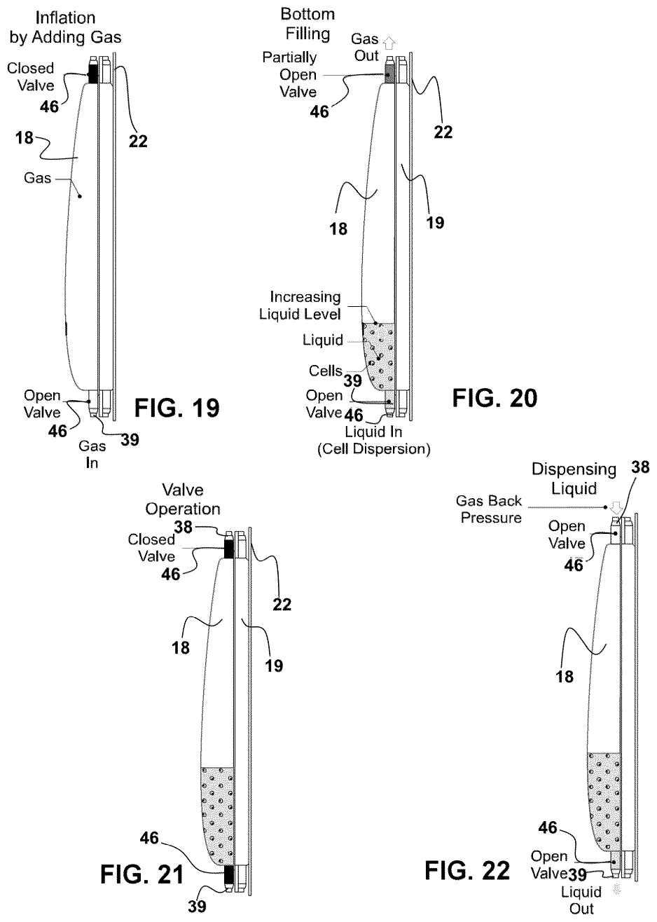

At least one first or upper conduit 36 having a valve actuator allows for controlled connection to an envelope first or upper tube 38. A second or lower conduit 40 may be provided with a controlled actuator to connect to a lower or second envelope tube 42 (FIG. 15). Where dual chamber fluid envelopes 18 are employed as in FIGS. 19-22, for example, there may be two first or upper conduits 36 and two second or lower conduits 40. The use of the terms upper and lower is for convenience, and it should be understood that the first conduit connected to the first tube and the second conduit, if provided, connected to the second tube, may be on the upper end, the lower end, or the side of the envelope.

In the software-enabled system of operation herein, the system provider will preferably capture control digital images 50, which may be sequential and correlate to time points during the aggregate duration of each processing regimen. By control digital images 50 is meant control images showing at least a correct volume of liquid in relation to the cell in the interior of the fluid envelope for the regimen, and a correct homogeneity or uniformity of the fluid and cell mixture within the fluid envelope for the regimen being controlled.

The control digital images 50 are digital images that are thereafter employable for comparison 54 to digital images 52 captured at time points during a processing regimen of the contents of the fluid envelopes 18, which correlate to the time points of the control digital images 50. The control digital images are stored 51 in electronic memory in databases correlating to each cell processing regimen, the system 10, using the processing unit 12, will perform.

The system provider will also determine the correct environmental parameters 56 for each processing regimen, which may be performed by the processing unit 12, at time points during the duration of such processing regimens. By correct environmental parameters, herein is meant at least one or a combination of air pressure within the fluid envelope 18, temperature within the fluid envelope, and gas composition within the fluid envelope 18 at time points during the duration of the processing regimen being controlled by the system.

The determined environmental parameters 56 and captured environmental parameters 58 for each processing regimen to be performed by the processing unit, at time points during the processing regimen chosen, may be compared 60 to captured fluid container environmental parameters 56 taken at capture time points correlating to regimen time points of the stored correct environmental parameters 56. During remedial actions where a match is not discerned 59 the system can make adjustments to the determined environmental parameters 58 to bring them substantially into a match with the determined environmental parameters 56 for the time point measured. This is done by communicating gas or air to increase air pressure, heating the area around the envelop 18, or communicating heated fluid to the fluid envelop 18 to bring the determined parameters in compliance with the correct environmental parameters 56. If such is determined as not possible, then a technician may be informed.

Each processing regimen performed by the system 10 may also have preferred container movements which should be imparted to the fluid envelope 18 as the processing regimen continues through a duration thereof. The preferred fluid container movements over the duration of each processing regimen controlled by the system 10 may be stored in the container movement database 62 for use during each processing regimen for the processing unit 12 controlled by the system 10. Once stored in electronic data, the system may actuate the processing unit 10 as required to impart the preferred movements.

As noted, the system 10 may control the processing unit 12 for any of a plurality of processing regimens for cell or other processing. In order to give users the ability to determine and choose a desired processing regimen, the system may display a menu 64 thereof, such as on a touch display screen. The user may then input 66 the desired processing regimen from those available for the system to control.

While in some modes the system 10 may be configured to populate the interior cavity of the fluid envelope 18 with the cells and media and the like for a chosen processing regimen, the user may currently populate the fluid envelope 18 with the appropriate media and cells and the like for a chosen processing regimen 68, and couple the fluid envelope 18 to the processing unit 12.

Thereafter, the system 10 will control the processing unit 12 to communicate the movements 70 to the fluid envelope 18 required for the chosen processing regimen and which are stored in electronic memory.

During the processing by the system, at capture time points during the duration of a processing regimen, captured digital images may be captured 52 by the digital camera 30 or digital microscope 32. These captured digital images 52 may be compared to the control digital images 54 to ascertain that the processing regimen is proceeding correctly, wherein the captured digital images 52 are substantially the same as the control digital images 50.

Where a substantial match 56 is ascertained by software operating to compare the captured digital images to the control digital images 50, then the system will continue 56 the processing regimen. Where a match is not discerned 59, the system employs software operating to take remedial actions to bring the processing regimen back into correct operation, whereupon the captured digital images 52 can be compared at the next time point, to discern a match 56. The remedial actions may add fluid or manipulate the fluid envelope 18 physically, such as to break up clumps or impart required uniformity to the mixture, for example.

If a match 56 is discerned, the process would continue; if no match is discerned, or if no remedial action is stored in the electronic database to correct a mismatch at a time point, then the system 10 may contact or inform a technician to take remedial action.

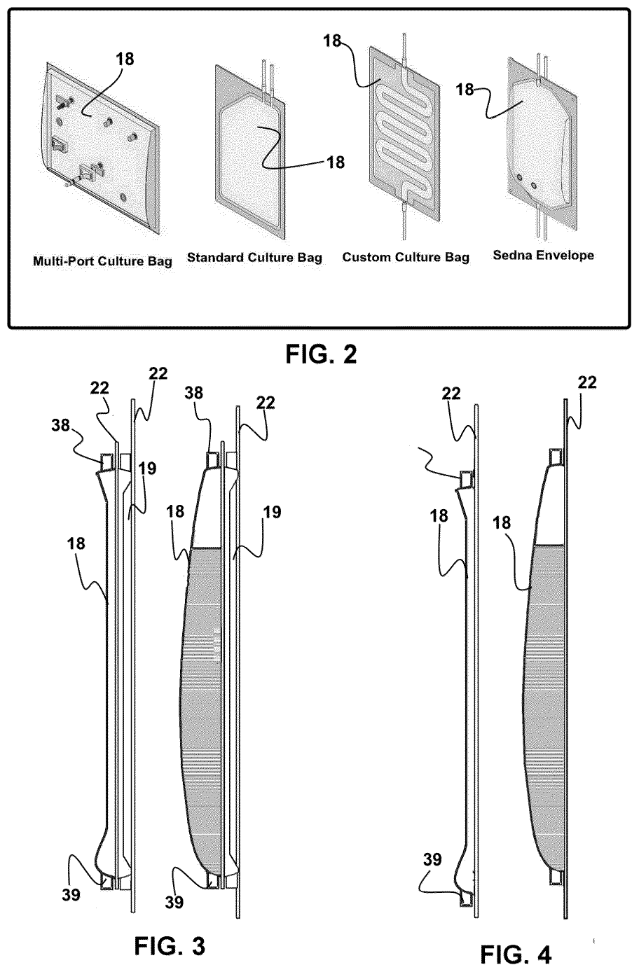

As noted, the system and processing unit 12 may be employed to perform processing regimens with any fluid envelope 18, which will operatively couple with the processing unit 12. As shown in FIG. 2, the differing fluid envelopes 18 are coupled to transparent planar backings 22, which may be coupled against an illuminated assembly 24 of the processing unit 12. Each has different upper and lower conduits, which can be matched to upper conduits 36 and lower conduits 40 and valve actuators therefor.

Shown in FIGS. 3-4 are side views of two preferred configurations of processing fluid envelopes 18. In FIG. 3, the processing fluid envelope 18 is dual-chambered and has a secondary envelope 19 and a fluid membrane 21 between the first processing fluid envelope 18 and the secondary envelope 19. The fluid membrane 21 can be configured to pass gas from the secondary envelope 19 to the fluid envelope 18. The first or upper tubes 38 and second or lower tubes 39 may be employed in combination with the upper and lower conduits and valve actuators controlling a flow therefrom, to input and evacuate fluid and gas from either a single fluid envelope 18 or the first fluid envelope 18 or secondary envelope 19 where there are two. This is similar to the single chamber processing envelopes shown in FIG. 4; however, there is no secondary chamber or membrane 21 in this configuration.

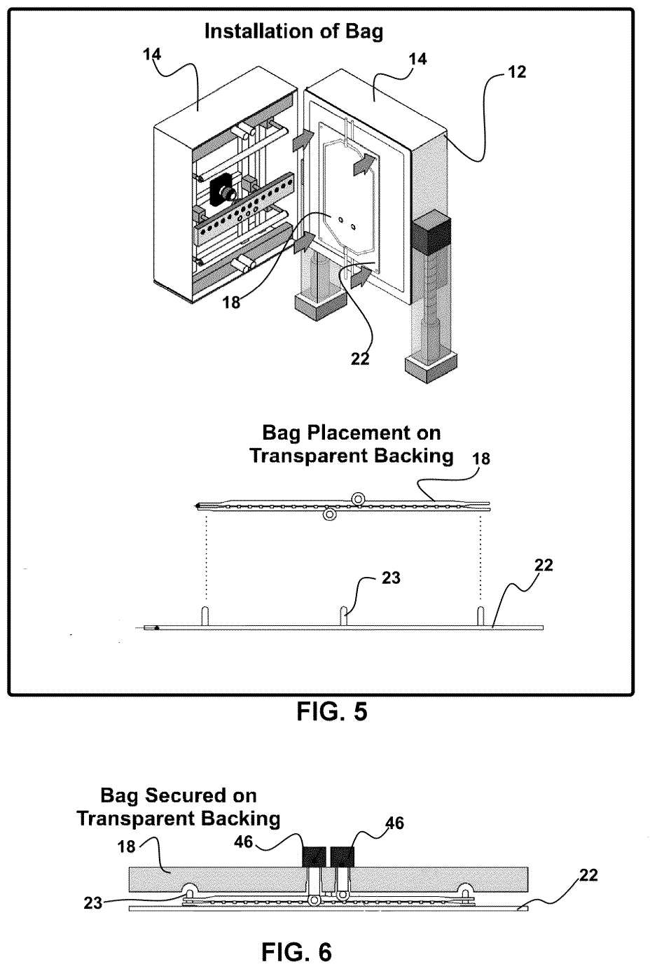

Shown in FIG. 5, the chosen type of fluid envelope 18 may be coupled to the planar backing 22 using mating connectors such as positioning pins 33 which couple with mating connectors such as apertures on the fluid envelope 18. The assembly of fluid envelope 18 and planar backing 22 is then hung or otherwise positioned within the processing unit 12 to place the substantially transparent planar backing 22 adjacent to the illumination assembly 24.

FIG. 6, is depicts a sectional view through a fluid envelope 18 showing valve actuators 46, which are electronically controlled by software operating to control air and fluid flows into the fluid envelope 18 through the envelope tubes 38 and 39.

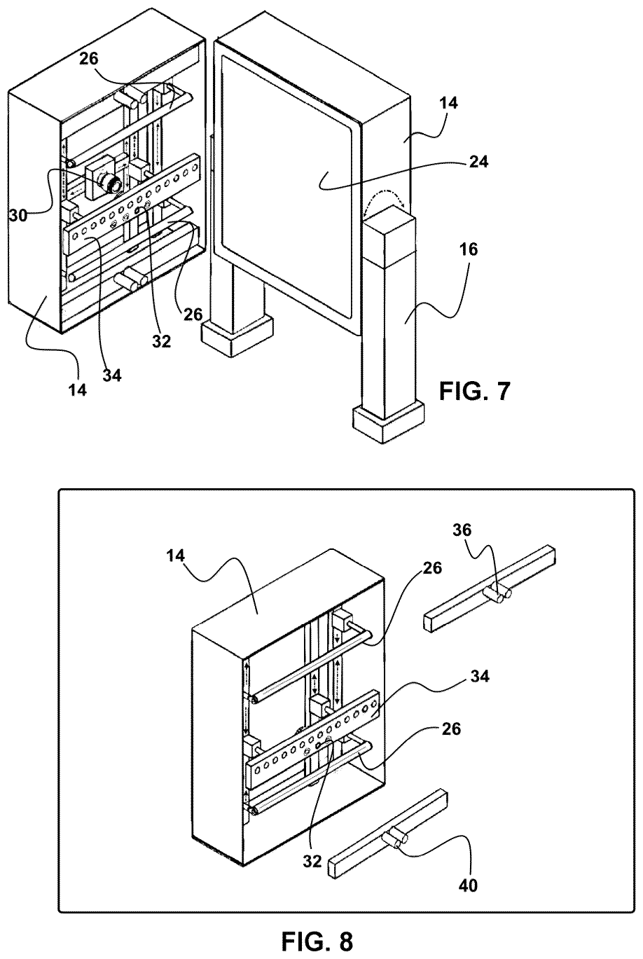

Shown in FIGS. 7-8 are various components of the processing unit 12. As shown, the housing 14 has two rotationally engaged sections which allow for the fluid envelope 18 to be hung or otherwise positioned adjacent the illumination assembly 24, such as an LED panel, and the two sections of the housing 12 are closed to position the fluid envelope 18 within an interior cavity of the housing 14.

The gas or, in some cases, fluid may be communicated to and from the interior of the fluid envelope 18, through the upper conduits 36 and lower conduits 40, which are engaged to manifolds which may be connected to the fluid or gas required for a particular processing regimen.

As noted above, the digital camera 30 is on a powered track 28, which allows for movement thereof as needed by the software operating the system. The microscopic camera 32 is recessed into a translating paddle 34, whereby the paddle 34 may contact against one side of a fluid envelope 18 and sandwich it between the paddle 34 and the illumination assembly 24 to capture digital images. The rollers 26, as also noted above, are engaged with powered tracks 28 whereby the software operating the system may position them as needed in contact against one side of the fluid envelope 18 to sandwich it between the rollers 26 and the illumination panel 34 and planar backing 22.

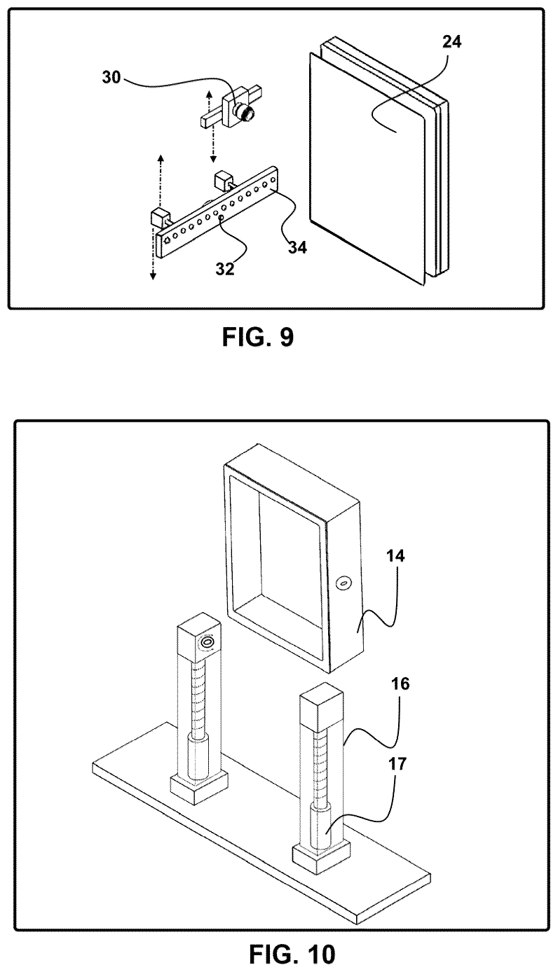

In FIG. 9, the imaging system's main components are shown. The illumination assembly 24 provides illumination or light which may be transmitted through the substantially transparent fluid envelope 18 and any liquid therein, to allow for the capture of digital images by both the camera 30 and the microscope 32. Both the camera 30 and microscope 32 have mounts engaged with tracks to allow for controlled movements of the camera 30 and microscope 32 across the surface of an envelope.

The rotational components of the system are shown in FIGS. 10-12. In FIGS. 10-11, the housing 14 is shown having a rotational coupling with the support 16, such as the depicted posts. A controllable motor 17 is in a geared or other powered coupling to impart controllable rotation to housing 14 to rotate and hold it in different positions, such as shown in FIG. 11. By controllable rotation herein is meant that using a controllable motor 17 or other powered rotation, a desired angle of the horizontal or vertical positioning of the housing can be provided, such as, for example, ninety degrees from a flat support surface or parallel to the flat support surface, and the motor energized to move the housing to that angle.

The controlled rotation and positioning of the housing 14 allows for the controlled rotation and static positioning of the fluid envelope 18 during the processing regimen. As noted, these individual movements and positioning may be associated with any of the processing regimens and the system employing software operating to the task, can rotate or rock the housing 14 to thereby rock and rotate the fluid envelope 18 mounted therein, to different moving and static positioning.

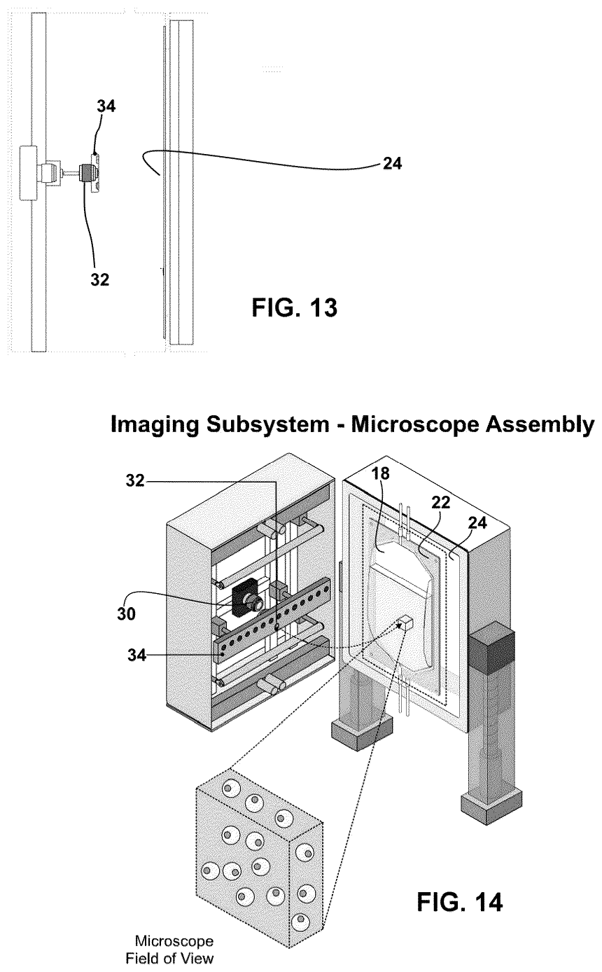

The translatable paddle 34, which carries the microscopic camera 32, is depicted in another view in FIGS. 13 and 14. As noted, the paddle 34 is adapted to press against the fluid envelope 18 and sandwich it between the illumination assembly 34 to allow for better microscopic imaging of the fluid within the fluid envelope 18. The field of view of the microscopic camera 32 is depicted in FIG. 14. As can be discerned the cells and fluid in the envelope may be microscopically photographed to digital images for the noted comparisons during the system control of a processing regimen.

Shown in FIGS. 15-18 are various configurations of the fluid envelope 18 that software operating to the task in the system can control. As shown in FIG. 15, showing empty envelopes, the single or dual chamber envelopes may be employed, or any other fluid envelope 18 that is configured to be operatively coupled to the illumination assembly or processing unit 12.

Initially, the fluid envelope 18 may be filled with gas, such as shown in FIG. 16-17, where the gas is input through upper and lower envelope tubes 38 and 39 from the upper conduits 36 and secondary or lower conduits 40. The flow of gas and fluid is controlled by valve actuators 46, which are controlled by software of the system operating to the task of filling and emptying gas and fluid to and from the fluid envelopes 18 as needed for each processing regimen. As shown in FIG. 16, gas may be communicated through a lower envelope tube 39 while the upper envelope tube 38 is closed by the valve actuator 46. Air in the interior of the fluid envelope 18 exits through a partially closed upper envelope tube 38 by partially closing the valve actuator 46. Once the envelope is filled with gas at a pressure as in FIG. 18, the actuator valve 46 is closed.

As one skilled in the art can discern, with actuators 46 on each upper and lower envelope tube, the system can easily control the actuators to vent air and fluid and add air or gas and fluid as needed for each processing regimen. Some of the different system-controlled operations to fill and empty the fluid envelopes 18 are shown in FIGS. 19-30.

FIG. 19 shows the adding of gas from a lower end of the fluid envelope 18 with the valve actuator 46 on the upper end closed to form an enlarged interior chamber filled with gas. Liquid is shown being communicated to the interior chamber through the bottom end of the fluid envelope 18, with the valve actuator on the lower end opening the lower tube 39 while the valve actuator 46 opens the upper tube 38 path to vent gas during filling. As in FIG. 20, the fluid remains in the interior chamber with a volume of gas once the valve actuators 46 are both closed, as in FIG. 21.

To drain liquid from the interior chamber of the fluid envelope 18 through the lower end thereof, the lower valve actuator opens, allowing fluid to drain through the lower envelope tube 39 while concurrently the valve actuator 46 on the upper envelope tube 38 opens to allow gas or air into the interior chamber.

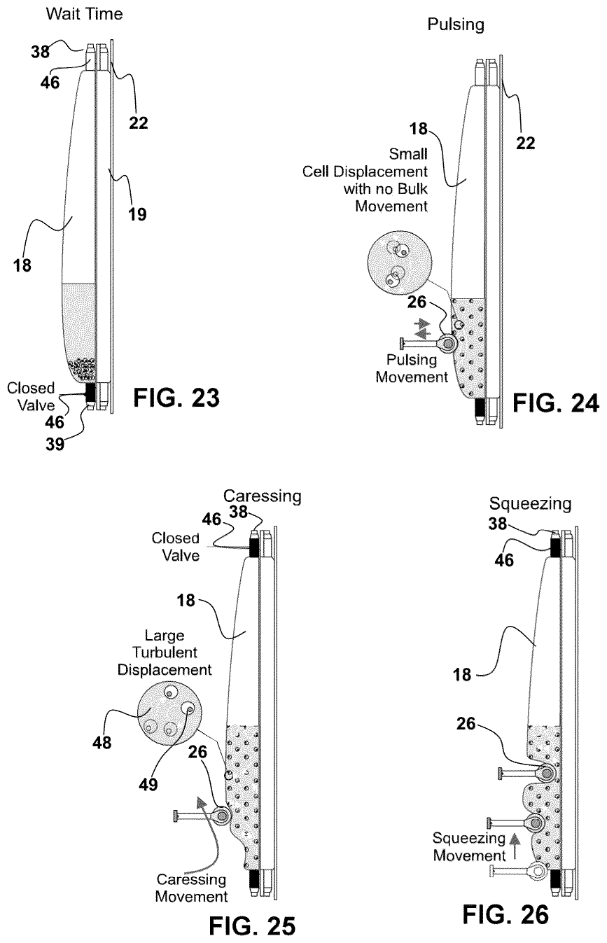

Shown in FIG. 23, once fluid and cells have been communicated into the interior chamber of the fluid envelope 18, a static positioning thereof allows for a wait time, which allows for a settling of the solids and cells 48 within the liquid 49. FIGS. 24-29 show different configurations available for the interior chamber of the fluid envelope 18 using the rollers 26 during processing.

In a pulsing or in-and-out translating movement of a roller 26, as in FIG. 24, the fluid 48 and solids 49, such as cells, may be mixed and the cells 49 disbursed in the liquid 48.

FIG. 25 depicts the controlled caressing movement of a roller 26 as controlled by the system software. This movement is shown as a caressing curved path where the roller 26 is pushed to deflect the sidewall of the fluid envelope 18 and move the mixture of fluid 48 and cells 49 in a massage-like moment of inward, upward, and back outward at a higher elevation.

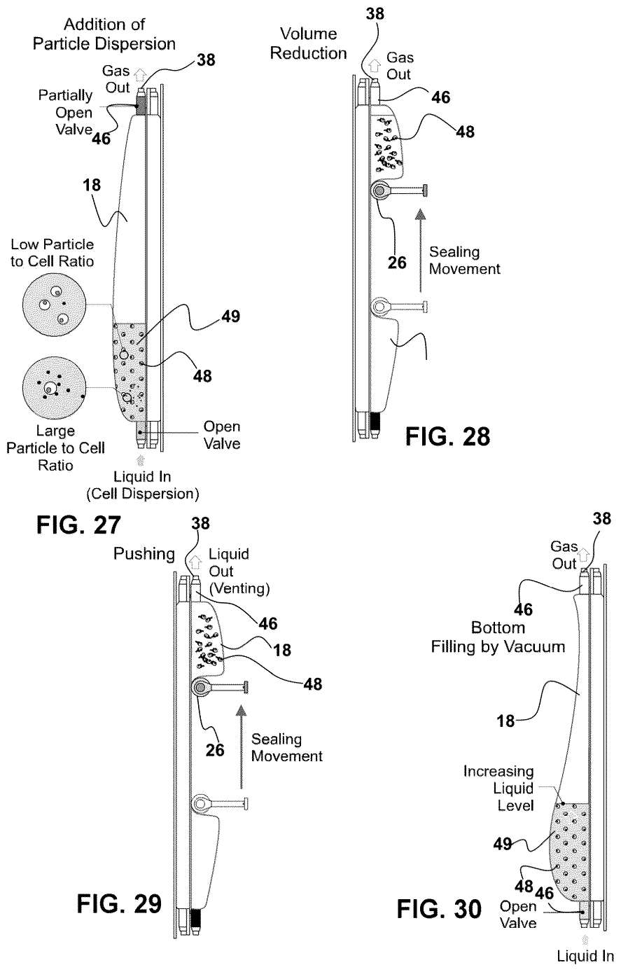

FIG. 26 depicts a squeezing movement enabled by the movement of the roller 26 to progressively deflect the sidewall of the fluid envelope 18 further and further into the expanded interior chamber.

Where there is a low particle to cell ratio within the contents of the fluid 48, as in FIG. 27, liquid 48 may be added from the lower end of the fluid envelope 18 by opening the valve actuator 46 and inputting liquid under pressure while the upper valve actuator 46 of the upper tube 38 is open.

In another movement of the system, as in FIG. 28, the roller 26 may operate in a sealing movement. In this action, the roller causes the two sidewalls of the fluid envelope 18 to contact each other, while the roller 26 concurrently moves toward the upper end of the fluid envelope 18. During this movement, the valve actuator 46 controlling flow through the upper envelope tube 38 is opened.

The upward movement of FIG. 28 of the roller 26 may be continued to vent liquid 48 out of the interior chamber through the upper envelope tube 38.

The liquid 48 may also be communicated into the interior chamber of the fluid envelope 18 using a vacuum formed therein. Gas may be pulled from the upper envelope tube 38, creating a vacuum wherein fluid 48 may be drawn through the lower envelope tube 39 in the lower end, as in FIG. 30.

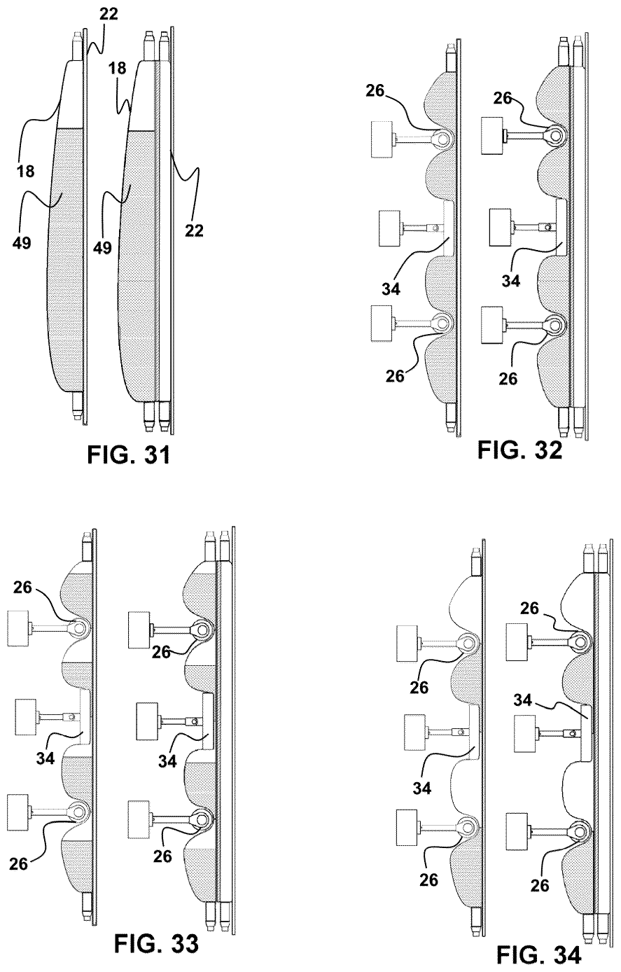

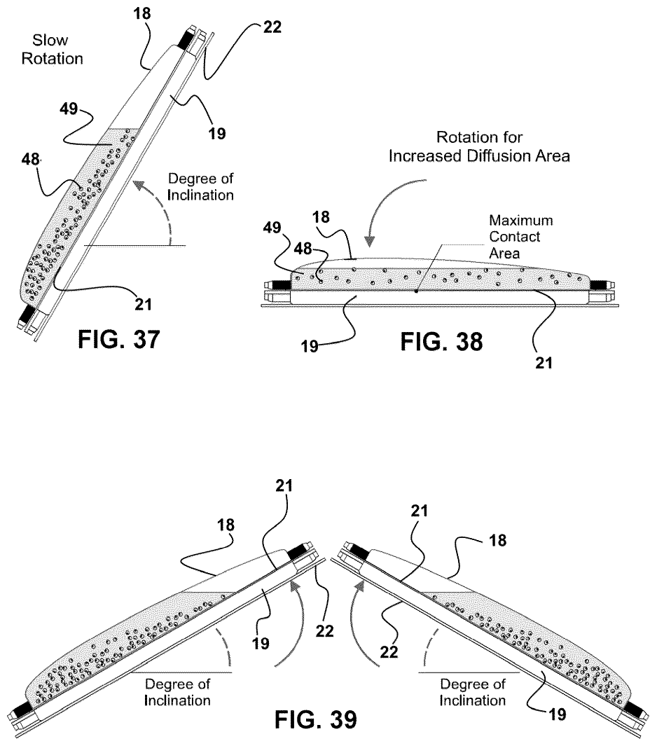

Shown in FIGS. 31-34 are configurations where the system employs both the paddle 34 and both rollers 26 concurrently, to divide the interior chamber of the fluid envelope 18 into a plurality of separated sections. The sections may be filled totally with fluid 48, as in FIG. 32, or may be partially fluid 48 and gas, as in FIG. 33, or may be sections full of fluid and sections full of gas as in FIGS. 32 and 34.

Where a single chamber fluid envelope 18 and also a dual chamber fluid envelope is employed, the system, as in FIGS. 37-39 can change the inclination angle of the fluid envelope 18 to increase or decrease to contact of fluid 48 against the membrane 21 and a gas-filled secondary chamber 19.

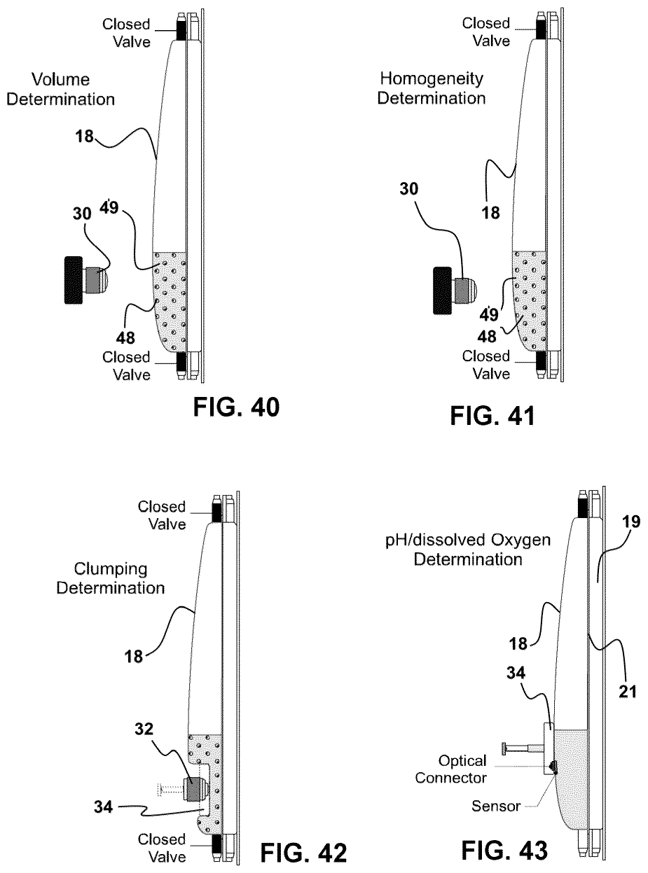

Actions of the camera 30 for volume determination is shown in FIG. 40, where the volume of the fluid 48 is determined using digital images. The camera 30 may also capture digital images for homogeneity determination, as in FIG. 41. These, as noted, may be compared with the library of control digital images for such to determine if processing at a time point is correct.

Digital images taken by the microscopic camera 32 mounted to the paddle 34 may be captured for the detection of clumping of the cells within the fluid 48. Such may also be compared to the library of pre-captured control images for such to determine a correct processing at a time point during the processing regimen.

In some cases, a sensor may be included within the interior cavity of the fluid envelope 18, as in FIG. 42. An optical connector on the paddle 34 may read the output of the sensor to determine pH/dissolved Oxygen.

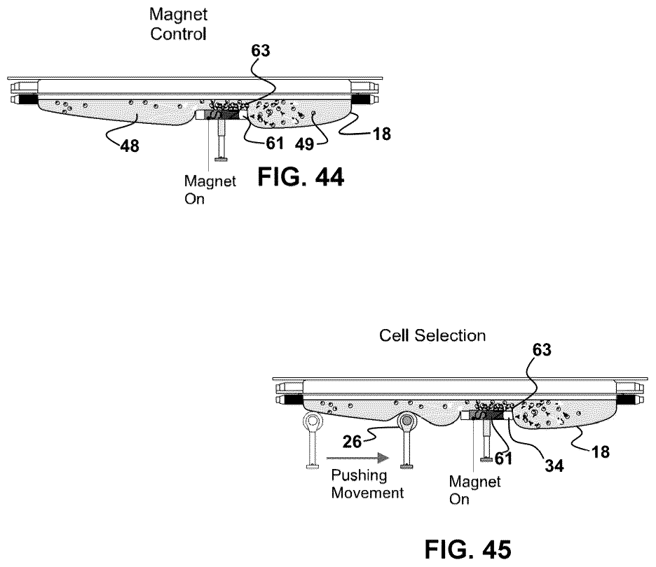

Finally, as in FIGS. 44-45, the system 10 may operate to control electromagnets 61 positioned on the paddle 34 as in FIGS. 44-45, to enable a paramagnetic bead within the interior chamber of the fluid envelope 18 to be attracted to and capture cells thereon. The electromagnetic beads can then be removed with the desired captured cells by translation of one of the rollers 26 across the fluid envelope 18 and toward a valve where the actuator 46 has been opened.

While all of the fundamental characteristics and features of the ATMP processing system have been shown and described herein, with reference to particular embodiments thereof, a latitude of modification, various changes and substitutions are intended in the foregoing disclosure and it will be apparent that in some instances, some features or steps of the disclosed system may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention herein disclosed.

What is claimed is:

1. A processing system for advanced therapy medicinal products comprising:
a housing, said housing having a first section and having a second section and having an interior cavity;
said housing in a coupling to a support;
a planar backing positioned in said interior cavity;
mating connectors configured for coupling of a processing envelope having an interior chamber, to a first side of said planar backing;
an illumination assembly positioned adjacent a second side of said planar backing opposite said first side thereof;
a camera positioned in said interior cavity for capturing digital images of the fluid contents of said interior chamber; and
whereby the contents of said interior chamber of said envelope, illuminated by light emissions from said illumination assembly, are captured in said digital images during a duration of processing thereof.

2. The processing system for advanced therapy medicinal products of claim 1, additionally comprising:
a microscopic camera positioned in said interior chamber, said microscopic camera for capturing digital microscopic images during said duration of processing; and
whereby said contents of said interior chamber of said envelope, illuminated by said light emissions from said illumination assembly, are captured in said microscopic digital images during said duration of processing.

3. The processing system for advanced therapy medicinal products of claim 1, additionally comprising:
said coupling of said housing to said support is a rotational coupling;
a motor, said motor actuable to impart controllable rotation to said housing to rotate and hold said housing to different vertical and horizontal positioning; and
whereby said processing envelope and said contents in said interior chamber thereof are positionable by said controllable rotation to said different horizontal and vertical positioning, during said duration of processing.

4. The processing system for advanced therapy medicinal products of claim 2, additionally comprising:
said coupling of said housing to said support is a rotational coupling;
a motor, said motor actuable to impart controllable rotation to said housing to rotate and hold said housing to different vertical and horizontal positioning; and
whereby said processing envelope and said contents in said interior chamber thereof are positionable by said controllable rotation to said different horizontal and vertical positioning, during said duration of processing.

5. The processing system for advanced therapy medicinal products of claim 1, additionally comprising:
at least one envelope tube having a tube passage in communication with said interior chamber; and
at least one conduit positioned in said interior cavity, said conduit having a conduit passage connectable to said tube passage to supply gas or fluid to said interior chamber.

6. The processing system for advanced therapy medicinal products of claim 2, additionally comprising:
at least one envelope tube having a tube passage in communication with said interior chamber; and
at least one conduit positioned in said interior cavity, said conduit having a conduit passage connectable to said tube passage to supply gas or fluid to said interior chamber.

7. The processing system for advanced therapy medicinal products of claim 3, additionally comprising:
at least one envelope tube having a tube passage in communication with said interior chamber; and
at least one conduit positioned in said interior cavity, said conduit having a conduit passage connectable to said tube passage to supply gas or fluid to said interior chamber.

8. The processing system for advanced therapy medicinal products of claim 4, additionally comprising:
at least one envelope tube having a tube passage in communication with said interior chamber; and
at least one conduit positioned in said interior cavity, said conduit having a conduit passage connectable to said tube passage to supply gas or fluid to said interior chamber.

9. The processing system for advanced therapy medicinal products of claim 1, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

10. The processing system for advanced therapy medicinal products of claim 2, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

11. The processing system for advanced therapy medicinal products of claim 3, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

12. The processing system for advanced therapy medicinal products of claim 4, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

13. The processing system for advanced therapy medicinal products of claim 4, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

14. The processing system for advanced therapy medicinal products of claim 6, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

15. The processing system for advanced therapy medicinal products of claim 7, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

16. The processing system for advanced therapy medicinal products of claim 8, additionally comprising:
at least one roller coupled with a powered track positioned in said interior cavity;
said roller positionable to a rolling contact against one side of said processing envelope to squeeze said envelope in a sandwiched positioning between said roller and said first side of said planar backing and a surface of said roller; and
said roller moveable by said powered track whereby said rolling contact imparts movement to said fluid contents.

17. A computer-enabled system for the production of advanced therapy manufactured products employing the processing system of claim 7:
a computer processor, and
a non-transitory, computer-readable medium communicably coupled to the processor and storing instructions that, when executed by the computer processor, cause the computer processor to perform operations comprising:
compiling a movement database of required fluid container movements over a duration for each of a plurality of processing regimens;
storing sequential control images of said fluid contents of said interior chamber, said sequential control images each depicting the fluid contents as correctly proceeding, at each of a plurality of respective time points during a duration of a respective processing regimen;
requesting an input for a chosen regimen from said movement database;
communicating said required fluid container movements for said chosen regimen to said housing having said fluid envelope coupled to said first side of said planar backing;
capturing said digital images of said fluid contents of said interior chamber at predetermined time points during said duration of said processing regimen;
comparing said digital images captured at said predetermined time points to said sequential control images correlating to said predetermined time points;
where said digital images captured at said predetermined time points match said sequential control images correlating to said predetermined time points, allowing the processing regimen to continue;
where said digital images captured at said predetermined time points are a mismatch to said sequential control images correlating to said predetermined time points, initiating remedial actions or communicating to a technician that a mismatch has occurred.
